(19) United States
(12) Patent Application Publication
(10) Pub. No.: US 2025/0134575 A1
(43) Pub. Date: May 1, 2025

(54) SYSTEMS AND METHODS FOR TREATING LABIAL HYPERTROPHY

(71) Applicants: John H. SHADDUCK, Menlo Park, CA (US); Michael HOEY, Menlo Park, CA (US)

(72) Inventors: John H. SHADDUCK, Menlo Park, CA (US); Michael HOEY, Menlo Park, CA (US)

(21) Appl. No.: 18/922,103

(22) Filed: Oct. 21, 2024

Related U.S. Application Data
(60) Provisional application No. 63/593,317, filed on Oct. 26, 2023.

(51) Int. Cl. A61B 18/12; A61B 18/00; A61B 18/04

(52) U.S. Cl. CPC A61B 18/12; A61B 2018/00625; A61B 2018/048

(57) ABSTRACT

Medical instruments and systems for applying energy to tissue, and more particularly relates to a vapor deliver system and methods for shrinking or modifying labial tissue with a flow of a condensable vapor that applies controlled thermal energy to targeted tissue.

### CROSS-REFERENCE TO RELATED APPLICATIONS

This is a non-provisional of U.S. Provisional application No. 63/593,317 filed Oct. 26, 2023, the entirety of which is incorporated by reference.

### BACKGROUND OF THE INVENTION

This invention relates to medical instruments and systems for applying energy to tissue, and more particularly relates to a vapor deliver system and methods for shrinking or modifying labial tissue with a flow of a condensable vapor that applies controlled thermal energy to targeted tissue.

In recent years, surgical treatments have become commonplace for altering the size, shape, and appearance of a female patient's vulva. The labia minora are typically the focus of concern. However, the entire anatomic region, including labia minora, labia majora, clitoral hood, perineum, and mons pubis, is known to be treated surgically. Labiaplasty is associated with high patient satisfaction and low complication rates. However, the basic labia minora reduction techniques such as edge excision, wedge excision, and central de-epithelialization all involve operative risks, perioperative care, and potential complications. See Sasson, D. et al., “Labiaplasty: The Stigma Persists”, Aesthetic Surgery Journal 2022, Vol. 42 (6) 638-643; Kalampalikis, A. et al., “Cosmetic Labiaplasty on Minors: A Review of Current Trends and Evidence”; Your Sexual Medicine Journal 2023 Vol. 35:192-195.

The present invention provides a less invasive procedure with needle-based vapor ablation of collagenous tissue and smooth muscle tissue to shrink labial tissue without the need for tissue excision. The vapor ablation method can also be used to reduce the volume of other anatomic regions of a patient's vulva.

### SUMMARY

Variations of the present disclosure include methods of treating labial hypertrophy. For example, such methods can include introducing a needle into a subject's labia; and delivering a flow of vapor through the needle to an interface with a labial tissue; wherein the vapor undergoes a vapor-to-liquid phase transition in the interface, thereby applying thermal energy sufficient to shrink labial tissue.

Additional variations of the methods described herein include delivery of vapor that shrinks at least one of collagenous tissue and smooth muscle tissue. The vapor can be any type of vapor, including, but not limited to water vapor.

Variations of the present disclosure include methods wherein the needle is advanced in the labial tissue dissecting a path in the labial tissue, followed by retracting the needle partially and delivering the vapor into the path thereby directing the application of energy to the labial tissue around the path.

Variations of the present disclosure methods where the needle is introduced more into a plurality of paths followed by delivering the vapor sequentially into the plurality of paths.

The methods of the present disclosure can include cooling an exterior surface of the labial tissue with a tissue cooling member or cooling opposing exterior surfaces of the labial tissue.

In an additional variation, the methods can include a tip of the needle that carries a bicap mechanism including a bipolar capacitance and further including the step of utilizing the bicap mechanism to differentiate between tissue types to thereby position the tip of the needle in targeted tissue.

In another variation, the method further includes advancing an elongate spacing member proximate the needle to create a space to direct the flow of vapor. The needle can be coupled to a handle where the vapor is generated in the handle by a heating mechanism that converts a liquid to the vapor.

In another example, the present disclosure includes methods of modifying tissue. For example, such methods can include introducing a needle into an anatomic structure of a female vulva wherein the anatomic structure is selected from the group of labia minora, labia majora, clitoral hood, perineum and mons pubis; and delivering a flow of vapor through the needle to an interface with targeted tissue of the anatomic structure; wherein the vapor undergoes a vapor-to-liquid phase transition in the interface thereby applying thermal energy sufficient to reduce a volume of the anatomic structure.

The vapor can shrink at least one of collagenous tissue and smooth muscle tissue. Alternatively, or in combination, the vapor can melt adipose tissue.

Variations of the present disclosure include a method wherein the needle carries a bicap (bipolar capacitance) mechanism and further including utilizing a bicap mechanism to differentiate between tissue types to thereby position a tip of the needle in targeted tissue.

Variations of the present disclosure include a system for thermal treatment of tissue including: a handle carrying a helical tubing assembly configured for resistive heating by an electrical source; a needle coupled to the handle dimensioned for insertion into a subject's labia; a pump coupled to a fluid source configured to delivering a fluid flow in the helical tubing assembly; a controller configured to control the pump and electrical source to convert a fluid flow to a flow of vapor in the helical tubing assembly; and bicap electrodes carried at a distal end of the needle coupled to the controller and adapted to differentiate between tissue types for positioning a needle tip in targeted tissue.

Additional variations of the present disclosure include a system wherein the needle has a proximal hub for detachable coupling to the handle, and electrical contacts in the proximal hub for coupling the bicap electrodes to the controller. The system can include a shaft of the needle that is surrounded by a thin elastomeric sheath.

### BRIEF DESCRIPTION OF THE FIGURES

FIG. 1 is a view of a medical system adapted for thermal treatment and shrinkage of targeted tissue, with a vapor generating mechanism and a vapor delivery needle configured for shrinking labial tissue in a human subject.

FIG. 2 is an enlarged view of a distal portion of the vapor delivery needle of FIG. 1.

FIG. 3 is a sectional view of the vapor delivery needle of FIG. 2 taken along line 3-3 of FIG. 2.

FIG. 4A is an illustration of a step in a method of shrinking a patient's labia, showing advancement of a vapor delivery needle in the labia.

FIG. 4B is a subsequent step of partially retracting the needle, leaving a dissected needle path in the labia.

FIG. 4C is a subsequent step showing the delivery of vapor from the needle tip, which conducts heat to tissue about the dissected needle path.

FIG. 4D shows the change in the profile of the labia and a subsequent step showing vapor delivery in another dissected needle path.

FIG. 4E illustrates a desired final change in the profile of the labia following shrinkage of collagenous tissue and smooth muscle tissue in the labia.

FIG. 5 is a view of another variation of a vapor delivery needle with a thin elastomeric sheath to prevent backflow of vapor along the needle shaft.

FIG. 6 is a view of another variation of vapor delivery needle with a plurality of vapor ports.

FIG. 7 is a view of another variation of vapor delivery needle.

FIG. 8 is a view of a polymeric curved vapor delivery needle.

FIG. 9 is a view of another variation of vapor delivery needle with an extendable member adapted for deployment in a dissected needle path for directing vapor flow.

FIG. 10A is a sectional view of a vapor delivery needle of the type shown in FIG. 9 being deployed in a dissected needle path.

FIG. 10B is a sectional view of a vapor delivery needle similar to FIG. 10A with two extendable members deployed in a dissected needle path.

FIG. 11 is a view of a method of the invention using a cooling member to cool a surface of a patient's labia during a vapor tissue shrinking treatment.

FIG. 12 is a view of a method similar to that of FIG. 11, with first and second cooling members positioned on opposing surfaces of a patient's labia during a vapor tissue shrinking treatment.

### DETAILED DESCRIPTION

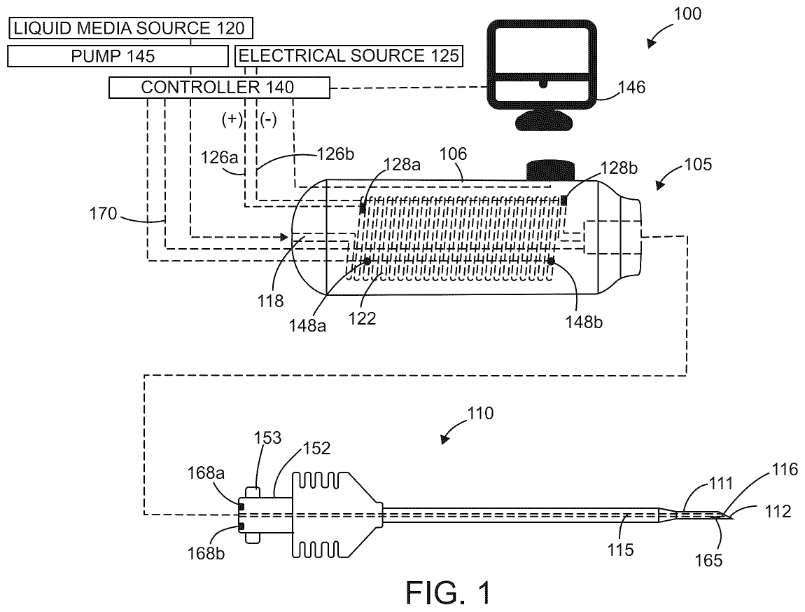

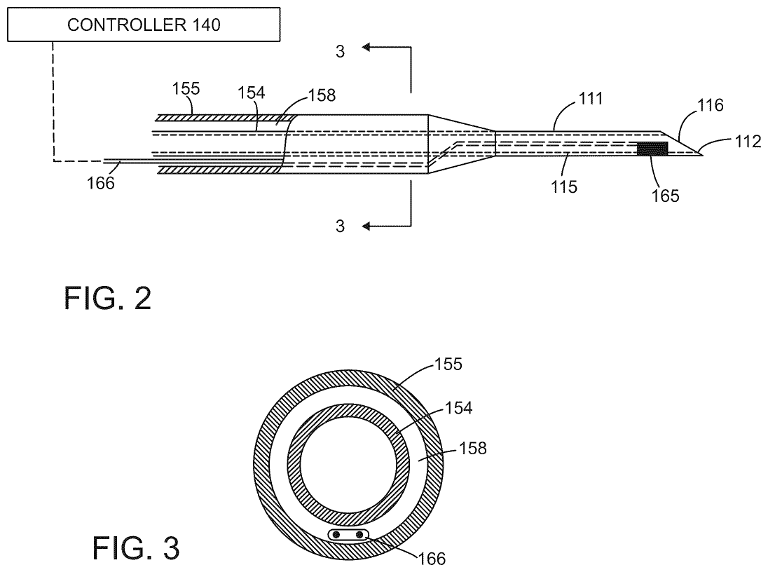

FIGS. 1 and 2 illustrate a variation of the invention that is configured for shrinking labial tissue and other soft tissues that utilize the application of energy from a condensing vapor that releases the heat of vaporization to tissue from a needle. In a variation, the system 100 of FIG. 1 comprises a vapor delivery device 105 with a handle 106 that is coupled to a detachable vapor needle delivery assembly 110 with elongated needle 111 having a distal end comprising a sharp needle tip 112. In this variation, the needle 111 has a vapor delivery lumen 115 that extends to an outlet 116 in the sharp tip 112. As will be described below, other variations of needles can have a plurality of outlets along an outer surface of a needle tip.

In the variation of FIG. 1, the handle 106 carries an electrical heating mechanism for applying energy to a flow of liquid media from a liquid media source 120, typically sterile water, in a flow channel 118 in the handle. In FIG. 1, helically formed metal tubing 122 carries the interior flow channel 118. An electrical source 125 is connected to electrical leads 126a and 126b that are conductively coupled at points 128a and 128b to opposing ends of the helical tubing 122 for resistively heating the helical tubing. The electrical source 125 is operatively coupled to a controller 140 for controlling energy delivery from the electrical source 125. The controller 140 also controls a pump 145 that is configured to pump the liquid media from the liquid media source 120 into and through the helical tubing 122. In operation, the controller 140 is adapted to precisely control the flow rate of liquid media and the temperature of the resistively heated helical tubing 122 to convert the liquid media to a vapor media in the helical tubing to thereby provide a high-quality vapor entering and exiting the outlet 116 in the needle tip 112.

In the variation of FIG. 1, it can be understood that the liquid media source 120, pump 145, and controller 140 are carried within a console that is coupled to the handle by a flexible conduit. In a variation, a display 146 is coupled to the controller, which can be a touchscreen display for selecting operating parameters of the system and for displaying alerts. The system 100 comprises a flow-based vapor delivery system, and typically, pump 145 comprises syringe pump known in the art that uses a stepper motor operatively coupled to controller 140 that allows for very precise control of flow rates of liquid media into the heating mechanism comprising the helical tubing 122. In a variation, the interior flow channel 118 of the helical tubing 122 has a diameter between 0.02″ and 0.10″ and a flow channel length of between 20 cm and 200 cm. The outside diameter of the helical tubing 122 as an assembly can be from 5 mm to 20 mm. In a variation, the helical tubing 122 can be formed of a stainless steel, Inconel, or any other suitable resistively heatable metal.

In a typical variation, the helical tubing 122 carries at least one temperature sensor coupled to the controller 140 and is shown in FIG. 1 with two temperature sensors 148a and 148b at proximal and distal ends of the helical tubing 122. In another variation, a third temperature sensor (not shown) is carried in a medial portion of the helical tubing 122. The plurality of temperature sensors are adapted to send temperature signals to the controller 140, and thereafter, the controller algorithms, in response to the temperature signals, modulates operation of the pump 145 and its flow rate and/or the energy delivered from electrical source 125 to insure the generation of high-quality vapor is provided by the heating mechanism which then results in the desired calories/second delivered from the needle 111.

It can be understood that the design parameters of the pump 145 and fluid inflow rates, the heating mechanism, and the electrical source 125 are inter-related, and in general, a typical system 100 is designed to provide a selected calories/second rate of applying energy to tissue that is optimal for the tissue shrinking procedure. In general, the inter-related design parameters include (i) ml/min of liquid media flow within the helical tubing 122 which further is dependent on flow channel diameter, flow channel length, and flow pressure; (ii) the power delivered by the electrical source 125 which further relates to helical tubing design and materials; and ultimately results in a selected vapor quality, i.e., the percent of the flow exiting a needle tip 112 that is phase changed to pure vapor as opposed to non-phase changed liquid droplets. In a variation, system 100 provides a flow of vapor that is greater than 90% pure vapor and further provides an ultimate conversion efficiency of electrical energy to vapor energy of at least 60%.

In one variation, the system includes an electrical source 125 that delivers at least 100 W, uses water as a liquid media source with a pump 145 providing a flow rate of between 1 ml/min and 5 ml/min into a helical channel having a diameter of 0.05″ and a length 50 cm with the helical tubing assembly having a diameter of 10 mm.

Referring to FIG. 1, it can be seen that the handle 106, which carries the helical tubing 122, is re-useable and detachable from vapor needle delivery assembly 110, which has a proximal hub 152 with suitable fluid-tight fittings such as a J-lock 153, O-rings and the like known for fluid-tight, sealed couplings. In this variation, the needle assembly 110 is configured for single use and is disposable.

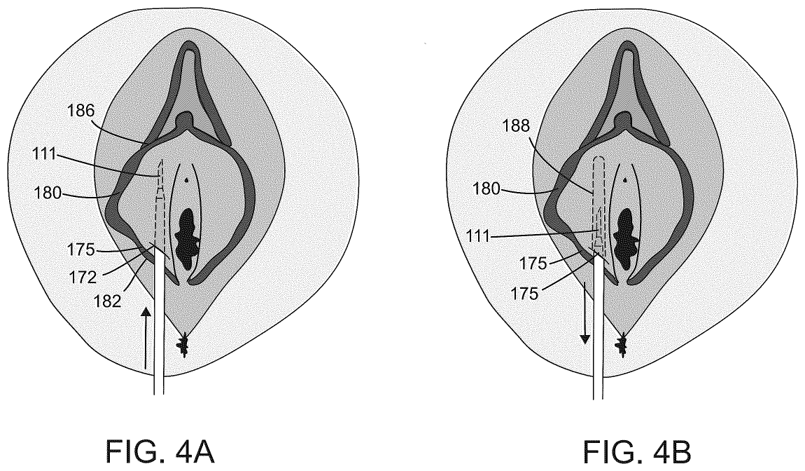

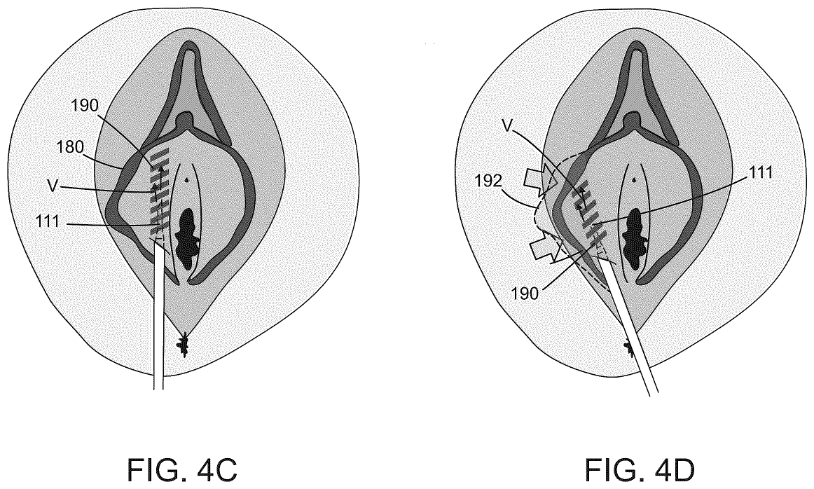

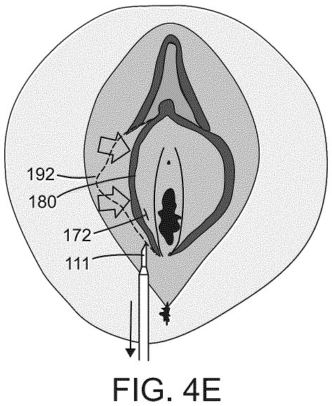

Referring now to FIGS. 2 and 3, the exposed needle tip 112 can comprise an 18 gauge to 27 gauge stainless steel needle having a suitable exposed length, which typically can be 2 mm to 10 mm for treating labial tissue, as shown in FIGS. 4A to 4E below. As can be best seen in FIGS. 2 and 3, needle 111 has a proximal region 154 that is insulated and is surrounded by an outer sleeve 155 that provides an insulative concentric space 158 around the proximal region 154 of needle 111, which can be an air space or most often a vacuum. The outer sleeve 155 can be a thin-wall stainless steel tube. In another variation, the insulative space 158 can comprise an aerogel or other insulative material, and the outer sleeve 155 can be a metal or polymeric material. The dimensions of the insulative space are designed to prevent the outer sleeve 155 from reaching a predetermined maximum temperature based on a particular vapor delivery interval during which vapor is flowing through the needle 111. A temperature sensor (not shown) can be provided at an exterior of the outer sleeve 155 and coupled to the controller 140 to send an alert to the operator of stop energy delivery through the system 100 or otherwise modulate operating parameters.

FIGS. 1 and 2 further show that the vapor delivery device 105 is configured with a bipolar capacitance mechanism or bicap electrodes 165 at the needle tip 112 as a component of a bicap system that functions to differentiate between different types of tissues based on electrical properties of tissue in contact with the electrodes. As can be seen in FIGS. 2 and 3, electrical lead 166 extends from the bicap electrodes 165 through the concentric space 158 around the proximal needle portion 156 and to the hub 152 of the needle assembly 110 (see FIG. 1) As can be seen in FIG. 1, electrical contacts 168a and 168b in hub 152 are coupled to cooperating contacts in handle 106 and an electrical cable 170 to couple the bicap electrodes 165 to the controller 140. As background relating to the bicap system, various tissues in the body have different electrical properties due to variations in their composition and water content. For example, muscle tissue, fat, and blood vessels have distinct electrical conductivities and capacitances. In a variation, the pair of bicap electrodes 165 are in close proximity, with a first electrode functioning as a transmitter while the second electrode functions as a receiver. The transmitter electrode emits a low-energy electrical signal, typically in the form of an alternating current, into the engaged tissue, and the electrical signal interacts with the tissue's electrical properties, primarily capacitance. The response is influenced by the specific electrical properties of the tissue, such as its dielectric constant and capacitance. The receiver electrode coupled to the controller 140 measures the response of the tissue, and controller algorithms can then evaluate the changes in the electrical signal and its phase to differentiate between tissue types. The results can be displayed in real-time on the display 146, or an audible alert can be provided to indicate to the physician that the needle tip 112 is in the targeted tissue.

In the treatment of labial hypertrophy, the tissue targeted for shrinkage and de-bulking consists of smooth muscle tissue, collagen strands, and other tissues deeper than the mucosal and dermal layers. The outer dermal layers are not targeted for thermal treatment, as dermal layers would not shrink substantially, and such dermal tissues contain a higher density of nerves, which should not be ablated. Anatomical studies report that nerve density/mm2 of labial is higher in the superior and inferior outer labia portions and the superior and inferior middle subunits of the labia minora. The innermost layer minora has slightly lower nerve density relative to the middle and outer parts, as the cutaneous nerve bundles originate from superior to inferior and tend to course more along the most outer edge of the labia minora. See Kelishadi, S. et al., “The Safe Labiaplasty: A Study of Nerve Density in Labia Minora and Its Implications”, Aesthetic Surgery Journal, 2016, Vol 36 (6) pp. 705-709. Thus, the bicap system can be adapted to identify smooth muscle and collagenous tissue to locate the needle tip therein and away from dermal tissue and tissue with higher nerve density to thereafter apply energy with the vapor delivery device 105.

Now, turning to FIGS. 4A to 4E, several steps of a method corresponding to the invention are illustrated. In FIG. 4A, the physician inserts the vapor delivery needle 111 at an entry point 172 in an inferior region 175 of the labia 180 inward of the labial edge 182 and advances the needle 111 in the superior direction. As the needle tip 112 is advanced, the bicap system can indicate when the tip of the needle 111 reaches dermal tissues in the superior edge 186 of the labia. By this means, the physician will be alerted to prevent penetration of the needle through the exterior of the labia 180. FIG. 4B then illustrates the physician partly retracting the needle 111 to thereby provide a dissected pathway 188 in the labia 180.

FIG. 4C then illustrates the physician actuating the vapor delivery device 105 to deliver vapor indicated at arrows V for a period of 1 to 10 seconds, and more often from 2 to 8 seconds, to thereby deliver energy to the labial tissue, where the vapor V will conductively heat tissue around the wall of the dissected path 188 (FIG. 4B). The thermally treated area is indicated as a hatched area 190 in FIG. 4C, which will instantly shrink collagenous and smooth muscle tissue to shrink the profile of the labia away from the original labia profile indicated at 192 in FIG. 4D. Following vapor delivery, the physician typically would redirect the tip of the needle 111 in another path, as shown in FIG. 4D and repeat the vapor delivery steps a second time. It should be appreciated that physicians may treat the labia in a plurality of different paths and entry points, for example, from 1 to 6 paths, depending on the selected needle size, vapor deliver interval, cal/sec delivered, and the volume of tissue targeted for shrinkage.

FIG. 4E then illustrates the reduced profile of the labia 180 away from the original profile 192 following the procedure where collagen and smooth muscle shrinkage result in a modified volume and shape of the labia 180. Subsequently, the physician may treat the opposing labia in a similar manner. It should be appreciated that the physician may elect any suitable needle entry point or multiple needle entry points to complete a volume reduction procedure. Since the entry point 172 of the needle is small, it is not likely that stitches would be needed, but stitches also may be used.

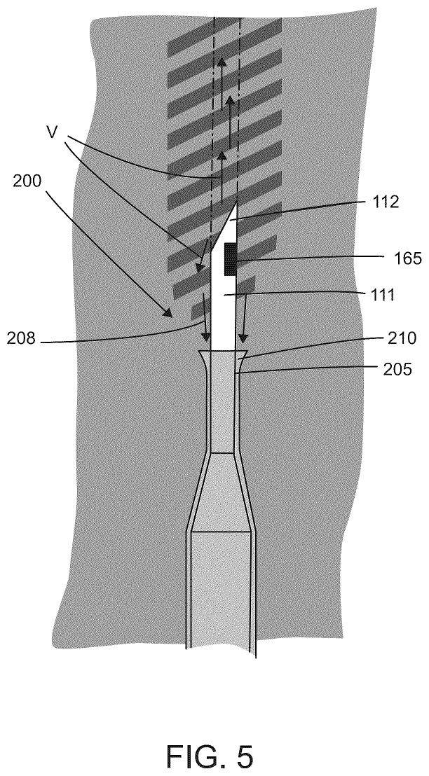

FIG. 5 illustrates a variation of a needle 200 with an additional component configured to prevent vapor V from flowing backward along the surface of the needle, which then could exit the needle entry point in the tissue. As can be seen in FIG. 5, the needle 200 carries a very thin elastomeric sleeve 205 around the shaft of the needle. Thus, when any vapor flows backward, as indicated at arrows 208, the backward flow along the needle shaft will migrate under a distal portion 210 of the elastomeric sleeve 205, causing it to bulge outward, which will capture any such backward flow of vapor V. Thus, an additional component of the invention comprises a thin wall elastomeric sleeve 205 covering, but not adhered to, the needle shaft for preventing backward vapor flows.

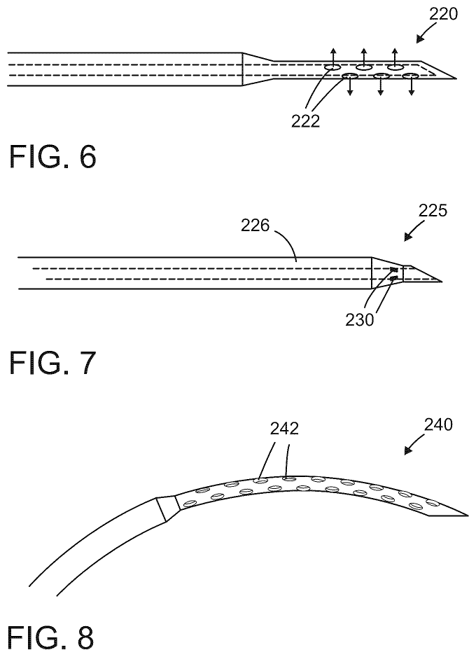

FIGS. 6, 7, and 8 illustrate other variations of a distal portion of vapor delivery needles. FIG. 6 illustrates a distal needle tip 220 configured with a plurality of vapor ports 222 extending along and around the circumference of the needle tip. In this variation, the vapor delivery can be directed 360 degrees around the tip, or the vapor ports 222 can be positioned only on opposing sides of the needle tip to direct vapor delivery in two directions. In this variation, the vapor needle 220 is adapted for convective heating of tissue where the vapor migrates extracellularly in tissue to ablate and shrink tissue rather than the vapor flowing in a dissected needle path, as shown in FIGS. 4A-4C. In this variation, the profile of the thermal treatment of tissue is controlled to a large extent by the arrangement of the vapor ports around the surface of the vapor needle 220.

FIG. 7 shows another variation, a needle shaft 225 that is largely encompassed by an outer insulating sleeve 226 to provide a vacuum chamber around the entire length of the needle. In this variation, the bicap electrodes 230 can be carried in a portion of the outer sleeve 226 rather than tip of the needle.

FIG. 8 illustrates another variation of an elongated needle tip 240 that again has a plurality of vapor ports 242 along and around the circumference of a curved needle tip. In a variation. The needle tip 240 is comprised of a polymer with a curved repose shape. The flexible polymer needle 240 can be extended outwardly from the straight tubular constraining sleeve (not shown). Such a curved needle can be used for shrinking labial tissue, as described above. Other variations can be configured for delivering ablative energy to a subject's labia majora or mons pubis to melt and reduce adipose tissues to reduce overall tissue volume, wherein lymphocytic reduction would occur over a time interval of 2 days to 2 weeks.

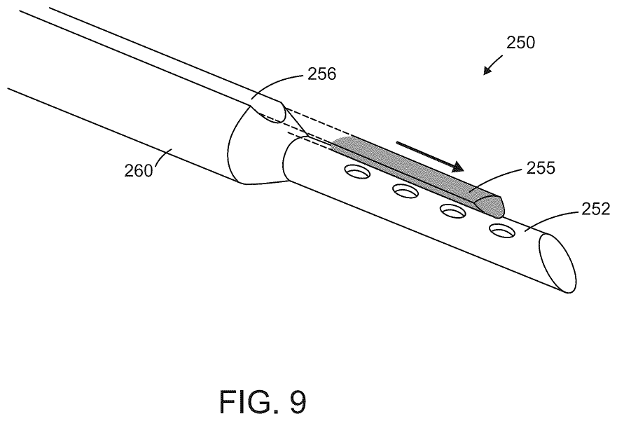

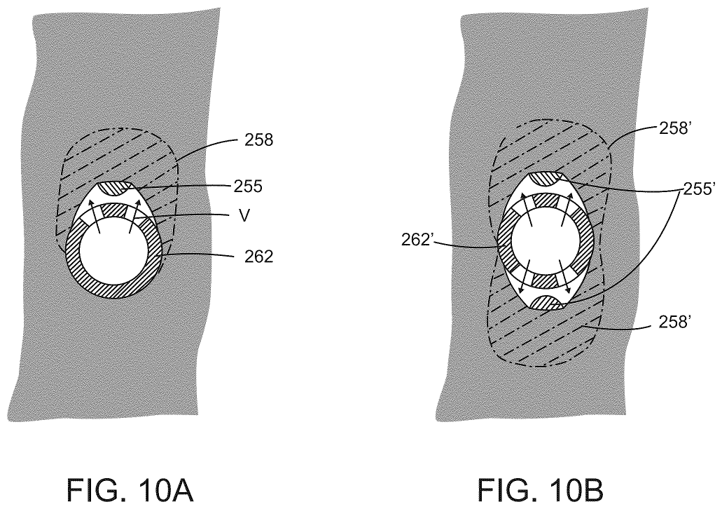

Now, turning to FIG. 9, another variation of a vapor delivery needle tip 250 is shown that is similar to the needle of FIGS. 1 and 2, except the variation of FIG. 9, includes an extendable member 255 that is carried in a recessed channel 256 in the outer concentric insulating sleeve 260. In a method of use, the extendable member 255 can be extended over the needle tip 262, as shown in FIG. 9 and the sectional view of FIG. 10A to provide a space for vapor to flow about the needle to direct the conductive thermal spread in tissue. In another method, the extendable member 255 is deployed following the insertion and partial withdrawal of the needle tip 262, as shown in FIGS. 4A and 4B. The extendable member 255 then extends beyond the distal tip of the needle to open and maintain the needle-dissected path for vapor flow therein. In such a variation, the method of maintaining a dissected needle path can allow for a directed thermal spread, as indicated at 258 in FIG. 10A about the needle path rather than convective heating via extracellular migration of vapor. The use of conductive heating about a dissected path is advantageous for directing ablation in a thin anatomic structure such as a subject's labia.

In another variation shown in FIG. 10B, the needle tip 262′ can carry a plurality of extendable members 255′ that can be extended to direct vapor flow as described above. In the sectional view of FIG. 10B, two extendable members 255′ are deployed to open the needle path to provide a directed thermal spread 258′. It should be appreciated that any number of extendable members can be used to open the needle path. In such variations, a method of the invention includes controlling vapor flow in a dissected path to allow for directed thermal conduction from vapor condensation and energy application to thereby provide an asymmetric thermal spread or ablation relative to the dissected path.

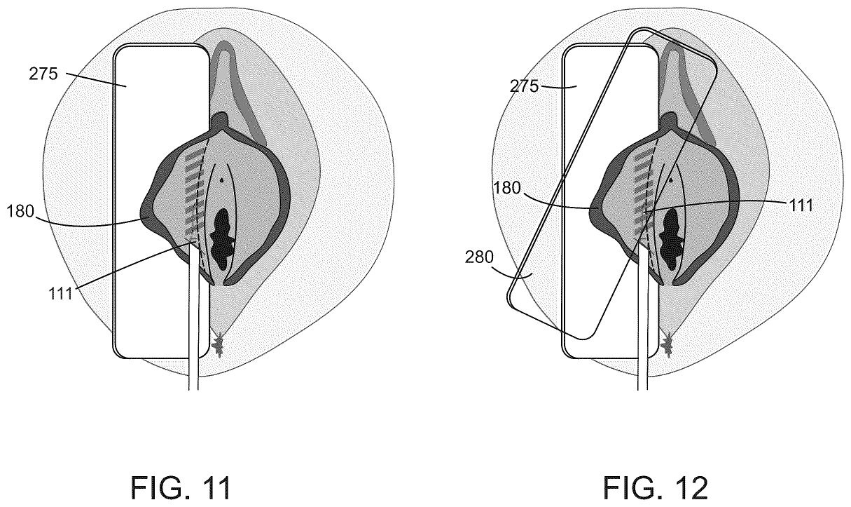

Now, turning to FIG. 11, another method corresponding to the invention is illustrated wherein a cooling member 275 is used to cool the exterior of the labia 180 prior to and during the delivery of vapor from a vapor delivery needle 111 of the type shown in FIGS. 1 and 2. In a variation, the cooling member 275 comprises a polymeric or metal material that has been cooled and is placed in a position to contact the exterior of the labia 180. The cooling member 275 can comprise any suitable solid or porous material that has been cooled, for example, in a refrigerated compartment for a suitable period of time. In a treatment procedure, the cooled member 275 is simply laid underneath the labia 180, which is gently positioned in contact with the cooling member. Thereafter, the steps as described can be followed to shrink the labia 180 while the cooling member 275 cools the exterior surface of the labia. By this means, the dermal tissue and nerve nerves of the surface of the labia 180 are further protected from thermal effects.

FIG. 12 illustrates a similar method corresponding to the invention wherein the first cooling member 275 is provided on a first surface of the targeted labia, and a second cooling member 280 is positioned on the opposing surface of this subject's labia 180. Thereafter, the physician delivers energy through the needle 111, as described above, to thermally shrink the interior tissues of the labia. By this means, the collagenous tissue and smooth muscle tissue in the interior of the labia can shrink and reduce the labia volume while the external dermal layers are protected. In a variation, the cooling member 280 can comprise a transparent polymer to allow visibility of the tissue and needle location.

In other variations (not shown), a cooling assembly can be an active device with a cooling fluid flowing in cooling channels of an applicator, Peltier elements, and the like. Also, a cooling applicator can include a mechanism for maintaining the labia in contact with the applicator, such as suction ports, adhering tapes, and the like. In another variation, a cooling assembly can include first and second opposing surfaces that are configured to engage and adhere to opposing sides of the labia and then to move the labia surfaces apart to stretch open the interior region of the labia for easier needle penetration.

And shown in FIG. 1 above, the system 100 uses resistively heated helical tubing 122 in handle 106 to generate water vapor. It should be appreciated that other variations are possible since the tissue treatments described above only need to deliver vapor for very short interval intervals, for example, one to 10 seconds, in a limited number of locations. Another system variation can include an entirely disposable handpiece with a disposable helical coil coupled to a needle. In such a variation, a remote console would still carry the fluid source, syringe pump, and electrical source. In another variation, a distal tip of a needle can be configured to generate vapor at the needle tip. In such a variation, typically, an elongate section of the tip would be resistively heated to generate the vapor. In yet another variation, a multiple-use handle could be provided that carried a battery as an electrical energy source, a cartridge fluid source, and a pump mechanism in the handle. Typically, such a variation would be coupled to a single-use, disposable needle. In other variations, the means of generating the flow of vapor can comprise at least one of resistive heating means, inductive heating means, radiofrequency (RF) energy means, microwave energy means, or photonic energy means.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications and patents cited herein are hereby incorporated by reference as if set forth in its entirety herein.

As for other details of the present invention, materials and manufacturing techniques may be employed as within the level of those with skill in the relevant art. The same may hold true with respect to method-based aspects of the invention in terms of additional acts that are commonly or logically employed. In addition, though the invention has been described in reference to several examples, optionally incorporating various features, the invention is not to be limited to that which is described or indicated as contemplated with respect to each variation of the invention.

Various changes may be made to the invention described and equivalents (whether recited herein or not included for the sake of some brevity) may be substituted without departing from the true spirit and scope of the invention. Also, any optional feature of the inventive variations may be set forth and claimed independently, or in combination with any one or more of the features described herein. Accordingly, the invention contemplates combinations of various aspects of the embodiments or combinations of the embodiments themselves, where possible. Reference to a singular item, includes the possibility that there are plural of the same items present. More specifically, as used herein and in the appended claims, the singular forms “a,” “and,” “said,” and “the” include plural references unless the context clearly dictates otherwise.

It is important to note that where possible, aspects of the various described embodiments, or the embodiments themselves can be combined. Where such combinations are intended to be within the scope of this disclosure.

We claim:

1. A method of treating labial hypertrophy comprising:
introducing a needle into a subject's labia; and
delivering a flow of vapor through the needle to an interface with a labial tissue;
wherein the vapor undergoes a vapor-to-liquid phase transition in the interface thereby applying thermal energy sufficient to shrink labial tissue.

2. The method of claim 1 wherein the vapor shrinks at least one of collagenous tissue and smooth muscle tissue.

3. The method of claim 1 wherein the vapor comprises water vapor.

4. The method of claim 1 wherein the vapor is delivered for 1 to 10 seconds.

5. The method of claim 1 wherein the needle is advanced in the labial tissue thereby dissecting a path in the labial tissue, followed by retracting the needle partially and delivering the vapor into the path thereby directing the application of energy to the labial tissue around the path.

6. The method of claim 4 wherein the needle is introduced more into a plurality of paths followed by delivering the vapor sequentially into the plurality of paths.

7. The method of claim 1 further comprising cooling an exterior surface of the labial tissue with a tissue cooling member.

8. The method of claim 6 further comprising cooling opposing exterior surfaces of the labial tissue.

9. The method of claim 1 wherein a tip of the needle carries a bicap mechanism comprising a bipolar capacitance and further comprising the step of utilizing the bicap mechanism to differentiate between tissue types to thereby position the tip of the needle in targeted tissue.

10. The method of claim 1 further comprising advancing an elongate spacing member proximate the needle to create a space to direct the flow of vapor.

11. The method of claim 1 wherein the needle is coupled to a handle and the vapor is generated in the handle by a heating mechanism that converts a liquid to the vapor.

12. A method of modifying tissue comprising:
introducing a needle into an anatomic structure of a female vulva wherein the anatomic structure is selected from the group of labia minora, labia majora, clitoral hood, perineum and mons pubis; and
delivering a flow of vapor through the needle to an interface with targeted tissue of the anatomic structure;
wherein the vapor undergoes a vapor-to-liquid phase transition in the interface thereby applying thermal energy sufficient to reduce a volume of the anatomic structure.

13. The method of claim 12 wherein the vapor shrinks at least one of collagenous tissue and smooth muscle tissue.

14. The method of claim 12 wherein melts adipose tissue.

15. The method of claim 12 wherein the needle carries a bicap (bipolar capacitance) mechanism and further comprising utilizing a bicap mechanism to differentiate between tissue types to thereby position a tip of the needle in targeted tissue.

16. A system for thermal treatment of tissue comprising:
a handle carrying a helical tubing assembly configured for resistive heating by an electrical source;
a needle coupled to the handle dimensioned for insertion into a subject's labia;
a pump coupled to a fluid source configured to delivering a fluid flow in the helical tubing assembly;
a controller configured to control the pump and electrical source to convert a fluid flow to a flow of vapor in the helical tubing assembly; and
bicap electrodes carried at a distal end of the needle coupled to the controller and adapted to differentiate between tissue types for positioning a needle tip in targeted tissue.

17. The system of claim 16 wherein the needle has a proximal hub for detachable coupling to the handle, and electrical contacts in the proximal hub for coupling the bicap electrodes to the controller.

18. The system of claim 16 wherein a shaft of the needle is surrounded by a thin elastomeric sheath.
